(19) United States
(12) Patent Application Publication
(10) Pub. No.: US 2007/0142478 A1
(43) Pub. Date: Jun. 21, 2007

(54) COMBINATION ANTIMICROBIAL COMPOSITION AND METHOD OF USE

(76) Inventors: Erning Xia, Penfield, NY (US); Hongna Wang, Fairport, NY (US); Srini Venkatesh, Pittsford, NY (US); Pramod Gupta, Pittsford, NY (US); Norman LaFrance, Rochester, NY (US)

Correspondence Address: Bausch & Lomb Incorporated, One Bausch & Lomb Place, Rochester, NY, 14604-2701, US

(21) Appl. No.: 11/610,901

(22) Filed: Dec. 14, 2006

Related U.S. Application Data
(60) Provisional application No. 60/752,413, filed on Dec. 21, 2005.
(60) Provisional application No. 60/752,455, filed on Dec. 21, 2005.
(60) Provisional application No. 60/760,510, filed on Jan. 20, 2006.
(60) Provisional application No. 60/760,880, filed on Jan. 20, 2006.
(60) Provisional application No. 60/782,478, filed on Mar. 15, 2006.
(60) Provisional application No. 60/830,319, filed on Jul. 12, 2006.
(60) Provisional application No. 60/830,326, filed on Jul. 12, 2006.

(51) Int. Cl. A61K 31/155

(52) U.S. Cl. 514/635

(57) ABSTRACT

Provided herein is a pharmaceutically acceptable composition comprising a biguanide containing antimicrobial agent and an additional antimicrobial agent. The invention further comprises topically administering the pharmaceutically acceptable composition to a patient in need of treatment.

### CROSS REFERENCE TO RELATED APPLICATION

This application claims priority to U.S. Provisional Patent Application Nos. 60/752,413 filed Dec. 21, 2005; 60/752,455 filed Dec. 21, 2005; 60/760,510 filed Jan. 20, 2006; 60/760,880 filed Jan. 20, 2006; 60/782,478 filed Mar. 15, 2006; 60/830,319 filed Jul. 12, 2006 and 60/830,326 filed Jul. 12, 2006; the contents of each being incorporated by reference herein.

### BACKGROUND OF THE INVENTION

1. Field of the Invention

This invention relates to the treatment of topical infections including nasal, ocular, otic, oral, vaginal, dermal and other topical infections.

2. Discussion of the Related Art

Ocular infections are not only uncomfortable conditions that require treatment, they often can result in permanent damage including corneal cysts and glaucoma, both of which can lead to a temporary or permanent loss of visual acuity and even blindness. Ocular infections include bacterial infections, viral infections, fungal infections and amoebal infections. These types of infections span four of the animal kingdoms and represent a wide range of genetic diversity.

For some time, biguanide antimicrobial agents have been used to preserve ophthalmic solutions and have been known for their relatively low toxicity in patients compared to other antimicrobial agents such as benkalkonium chloride. Biguanide antimicrobial agents include polyhexamethylene biguanide, chlorhexidine and alexidine.

To effectively preserve an ophthalmic composition, enough of the preservative is needed to prevent growth of a panel of bacteria, fungi and yeast. Typically, a product will contain about the lowest amount of a preservative required to accomplish the desired effect. Between about 0.5 ppm and about 1.5 ppm of a biguanide is needed to preserve most ophthalmic solutions.

Biguanide antimicrobial agents have also been used as disinfectant solutions for contact lenses. To be considered a disinfectant, a solution needs sufficient antimicrobial agent to kill a panel of bacteria and fungi over the shelf life of the product.

Disinfecting solutions containing biguanide antimicrobial agents include ReNu® Multiplus sold by Bausch & Lomb, Rochester, N.Y. ReNu® Multiplus is a multipurpose cleaning, conditioning and disinfecting solution for contact lenses that contains 3 ppm of polyhexamethylene biguanide.

Disinfecting solutions such as the one mentioned above are ophthalmically safe solutions. They are safe to administer to the eye of a patient. Contact lenses that have been rinsed with these solutions are placed in the eye. However, these solutions are not recommended for use as a medicament in the eye. There is no evidence to suggest that a multipurpose contact lens solution would be effective to treat ocular infection, such as viral infection.

Several studies have been conducted on the effectiveness of polyhexamethylene biguanide and/or chlorhexidine for treatment of Acanthamoebal keratitis.

In Schuster, et al., “Opportunistic Amoebae: Challenges In Prophylaxis And Treatment,” Drug Resistance Updates: Reviews And Commentaries In Antimicrobial And Anticancer Chemotherapy, vol. 7(1) pp. 41-51 (February 2004), Acanthamoeba keratitis, a non-opportunistic infection of the cornea, was found to respond well to treatment with chlorhexidine gluconate and polyhexamethylene biguanide, in combination with propamidine isothionate (Brolene), hexamidine (Desomodine), or neomycin.

In Rama et al., “Bilateral Acanthamoeba keratitis with late recurrence of the infection in a corneal graft: a case report,” European Journal of Ophthalmology, vol. 13 (3), pp. 311-4 (April 2003), recurrences of Acanthamoeba keratitis in both eyes were successfully treated with a combination of hexamidine and neomycin, and with polyhexamethylene biguanide respectively.

Anita et al., “Role of 0.02% polyhexamethylene biguanide and 1% povidone iodine in experimental Aspergillus keratitis,” Cornea, Vol. 22 (2), pp. 138-41, (March 2003) showed that polyhexamethylene biguanide (0.02%) is a moderately effective drug for experimental Aspergillus keratitis.

Sharma et al., “Patient characteristics, diagnosis and treatment of non-contact lens related Acanthamoeba keratitis,” British Journal of Ophthalmology, Vol. 84/10, pp. 1103-1108 (2000) illustrates the combination of polyhexamethylene biguanide and chlorhexidine.

Physicians frequently treat ocular infections with antibiotics. However, when the microbe causing the ocular infection is not a bacteria, antibiotics are not effective and the infection can worsen due to delay in providing effective treatment. Consequently, there is a need for a stable composition that is effective at treating a wide range of microbes. Additionally, it would be desirable to have a composition containing at least two agents that are effective at destroying microbial infection by different mechanistic pathways. The compositions prepared in accordance with this invention can be used advantageously as a nasal, otic, oral, vaginal, dermal or other topical composition as well as an ophthalmic one in the treatment of disease including disease of the eye, ear, nasal passages, vaginal canal or dermis.

### SUMMARY OF INVENTION

Provided herein are compositions for treating infectious disease. The compositions comprise a biguanide antimicrobial agent in an amount effective to treat infectious disease and a pharmaceutically acceptable carrier. Optionally, the pharmaceutically acceptable composition can comprise a biguanide antimicrobial agent and an additional antimicrobial agent with both present in an amount effective at treating microbial infections caused by either similar microbes or different microbes. The biguanide antimicrobial agent and the additional antimicrobial agent act synergistically or by different mechanisms making the treatment more effective for resistant strains of microbes than use of either agent alone. The present invention is effective at treating a variety of microbial infections such as bacterial, viral and fungal infections. Therefore, the present invention also includes a method of treating infectious disease. The method comprises administering a pharmaceutically acceptable composition comprising a biguanide antimicrobial agent to a patient infected with an infectious disease.

### DETAILED DESCRIPTION OF THE PREFERRED EMBODIMENTS

The active ingredients are used in the topically administrable therapeutic compositions for microbial eye disease as well as other topical disease such as nasal or otic disease in accordance with the invention (although such compositions are occasionally referred to herein as “ophthalmic” and words of similar meaning, use of this abbreviation does not exclude the application of the invention in the nasal, otic, oral, vaginal or dermal fields).

The term “composition” as used herein, refers to various forms of the compounds or compositions of the present invention, including solids such as powders, mixtures of powders and the like, ointments, gels, films, emulsions, suspensions as well as solutions.

The term “treating” refers to any indicia of success in the treatment or amelioration or prevention of an ocular disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an eye examination. Accordingly, the term “treating” includes the administration of the compounds or agents of the present invention to prevent or delay, to alleviate, or to arrest or inhibit development of the symptoms or conditions associated with disease. The term “therapeutic effect” refers to the reduction, elimination, or prevention of the disease, symptoms of the disease, or side effects of the disease in the subject.

The compositions of the invention herein have the following potential areas of application: bacterial inactivation, ophthalmic, general surgical preparation, etc. In addition, these compounds are also useful for the treatment of infections, such as acute or chronic Rhinosinusitis or other infections such as Otitis, Dermatitis, Bronchititis, Pneumonia's such as Pneumocystis carinii, infections of sex organs, such as Colpitis, Endometritis, Balnitis, infections of the gastrointestinal tract, such as Stomatitis, Oesophagitis, Enteritis, or infections of the urethra, such as Pyelonephrititis, Ureteritis, Cystitis, or Urethritis.

The compositions of the invention are stabilized to meet the requirement of being useable as compositions for the treatment or prevention of bacterial, microbial, spore, fungal and viral infections or contaminations.

The compositions of the present invention possess activity toward microbes, i.e., antimicrobial activity. As used herein, the term “antimicrobial” is meant to include prevention, inhibition, termination, or reduction of virulence factor expression or function of a microbe. “Prevention” can be considered, for example, to be the obstruction or hindrance of any potential microbial growth. “Termination” can be considered, for example, to be actual killing of the microbes by the presence of the composition. “Inhibition” can be considered, for example, to be a reduction in microbial growth or inhibiting virulence factor expression or function of the microbe.

As used herein, “microbe” or “microbial agent” is meant to include any organism comprised of the phylogenetic domains bacteria and archaea, as well as unicellular and filamentous fungi (such as yeasts and molds), unicellular and filamentous algae, unicellular and multicellular parasites, and viruses that causes disease in a subject. Accordingly, such microbial agents include, but are not limited to, bacterial, viral, fungal, or protozoan pathogens.

The present invention includes a composition for treating microbial infections. Typically, the composition comprises water, a biguanide antimicrobial agent in an effective amount and an additional antimicrobial agent in an effective amount. The biguanide antimicrobial agent and the additional antimicrobial agent are both present in an amount that is synergistically effective. The present invention is effective, in one embodiment, at destroying fungal infection but is also effective at destroying other secondary infections in the eye such as bacterial infections, amoeba infections and viral infections that are potentially present in the eye at the time of the fungal infection.

In one embodiment, the biguanide antimicrobial agent is selected from the group consisting of polyhexamethylene biguanide, chlorhexidine and alexidine.

By biguanide antimicrobial agent it is meant an antimicrobial agent that has two adjacent biguanide substituents in the molecule and antimicrobial properties in a pharmaceutically safe amount. Suitable biguanide antimicrobial agents include but are not limited to 1,1′-hexamethylene-bis[5-(p-chlorophenyl)biguanide] (Chlorhexidine) or water soluble salts thereof, 1,1′-hexamethylene-bis[5-(2-ethylhexyl)biguanide] (Alexidine) or water-soluble salts thereof, polyhexamethylene biguanide (PHMB) or combinations thereof.

In another embodiment, the biguanide antimicrobial agent is present in an amount that is a minimum of about 2 ppm and is a maximum of about 0.1 wt. % based upon the total amount of the composition.

When the formulation contains an additional antimicrobial agent the additional antimicrobial agent is selected from the group consisting of antibacterials, antivirals and antifungals and combinations thereof.

Antibacterial agents that are useful in combination with a biguanide antimicrobial agent of the present invention include but are not limited to antibiotics such as aminoglycosides (e.g., amikacin, apramycin, arbekacin, bambermycins, butirosin, dibekacin, dihydrostreptomycin, fortimicin(s), gentamicin, isepamicin, kanamycin, micronomicin, neomycin, neomycin undecylenate, netilmicin, paromomycin, ribostamycin, sisomicin, spectinomycin, streptomycin, tobramycin, trospectomycin), amphenicols (e.g., azidamfenicol, chloramphenicol, florfenicol, thiamphenicol), ansamycins (e.g., rifamide, rifampin, rifamycin sv, rifapentine, rifaximin), β-lactams (e.g., carbacephems (e.g., loracarbef), carbapenems (e.g., biapenem, imipenem, meropenem, panipenem), cephalosporins (e.g., cefaclor, cefadroxil, cefamandole, cefatrizine, cefazedone, cefazolin, cefcapene pivoxil, cefclidin, cefdinir, cefditoren, cefepime, cefetamet, cefixime, cefinenoxime, cefodizime, cefonicid, cefoperazone, ceforanide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, pivcefalexin), cephamycins (e.g., cefbuperazone, cefinetazole, cefininox, cefotetan, cefoxitin), monobactams (e.g., aztreonam, carumonam, tigemonam), oxacephems, flomoxef, moxalactam), penicillins (e.g., amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, meziocillin, nafcillin sodium, oxacillin, penamecillin, penethamate hydriodide, penicillin g benethamine, penicillin g benzathine, penicillin g benzhydrylamine, penicillin g calcium, penicillin g hydrabamine, penicillin g potassium, penicillin g procaine, penicillin n, penicillin o, penicillin v, penicillin v benzathine, penicillin v hydrabamine, penimepicycline, phenethicillin potassium, piperacillin, pivampicillin, propicillin, quinacillin, sulbenicillin, sultamicillin, talampicillin, temocillin, ticarcillin), other (e.g., ritipenem), lincosamides (e.g., clindamycin, lincomycin), macrolides (e.g., azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, miokamycin, oleandomycin, primycin, rokitamycin, rosaramicin, roxithromycin, spiramycin, troleandomycin), polypeptides (e.g., amphomycin, bacitracin, capreomycin, colistin, enduracidin, enviomycin, fusafungine, gramicidin s, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (e.g., apicycline, chlortetracycline, clomocycline, demeclocycline, doxycyc line, guamecycline, lymecycline, meclocycline, methacycline, minocyc line, oxytetracycline, penimepicycline, pipacycline, rolitetracycline, sancycline, tetracycline) and others (e.g., cycloserine, mupirocin, tuberin).

Also useful in combination with a biguanide antimicrobial are synthetic antibacterials such as 2,4-Diaminopyrimidines (e.g., brodimoprim, tetroxoprim, trimethoprim), nitrofurans (e.g., furaltadone, furazolium chloride, nifuradene, nifuratel, nifurfoline, nifurpirinol, nifurprazine, nifurtoinol, nitrofurantoin), quinolones and analogs (e.g., cinoxacin, ciprofloxacin, clinafloxacin, difloxacin, enoxacin, fleroxacin, flumequine, grepafloxacin, lomefloxacin, miloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin), sulfonamides (e.g., acetyl sulfamethoxypyrazine, benzylsulfamide, chloramine-b, chloramine-t, dichloramine t, n-formylsulfisomidine, n-(4)-β-d-glucosylsulfanilamide, mafenide, 4′-(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidochrysoidine, sulfamoxole, sulfanilamide, 4-sulfanilamidosalicylic acid, n-(4)-sulfanilylsulfanilamide, sulfanilylurea, n-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole) sulfones (e.g., acedapsone, acediasulfone, acetosulfone sodium, dapsone, diathymosulfone, glucosulfone sodium, solasulfone, succisulfone, sulfanilic acid, p-sulfanilylbenzylamine, sulfoxone sodium, thiazolsulfone) and others (e.g., clofoctol, hexedine, methenamine, methenamine anhydromethylene-citrate, methenamine hippurate, methenamine mandelate, methenamine sulfosalicylate, nitroxoline, taurolidine, xibomol).

In one preferred embodiment, the antibacterial agent is a compound selected from the group consisting of bacitracin, chloramphenicol, ciprofloxacin, erythromycin, gatafloxicin, gentamicin, levofloxacin, moxifloxacin, ofloxacin, polymzin B, sulfonamides, tobramycin, optical isomers, crystalline forms, salts, acids and bases of any of the foregoing and combinations thereof.

In one embodiment, the antibacterial agent is any compound that is disclosed in U.S. Pat. No. 5,447,926, which is incorporated herein by reference. In one embodiment, the antibiotic is of the following formula (I): 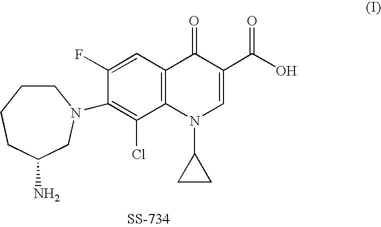
or its optical isomer, mixtures of SS-734 and its optical isomer enantiomers including racemic mixtures. In another embodiment, the antibiotic is in its salt form including inorganic salts and organic salts such as a chloride salt.

In one embodiment, the antibacterial agent is present in an amount that is effective at treating bacterial infections. Preferably, the amount of antibacterial agent is a minimum of about 1 ppm and/or a maximum of about 10 wt. %. More preferably, the amount of antibacterial agent is a minimum of about 10 ppm, 100 ppm, 1000 ppm, 0.1 wt. % and/or a maximum of about 5 wt. %, 2 wt. %, 1 wt. % and 0.1 wt. %.

Antiviral agents are also useful in combination with a biguanide antimicrobial. Antiviral agents include but are not limited to the agents selected from the group consisting of cidofovir, amantadine, rimantadine, acyclovir, ganciclovir, penciclovir, famciclovir, foscarnet, ribavirin, valcyclovir, trifluridine, fomivirsen, vidarabine, cyclosporine, valganciclovir, oseltamivir, adefovir, dipivoxil and zanamivir.

In one embodiment, the additional antiviral agent is present in an amount that is effective at treating viral infections. Preferably, the amount is a minimum of about 1 ppm and/or a maximum of about 10 wt. %. More preferably, the amount is a minimum of about 10 ppm, about 100 ppm, about 1000 ppm, about 0.1 wt. % and/or a maximum of about 5 wt. %, about 2 wt. %, about 1 wt. % and about 0.1 wt. %.

Antifungal agents are also useful in combination with a biguanide antimicrobial. Antifungal agents include but are not limited to the agents selected from the group consisting of polyene antifungal agents, imidazole antifungal agents, triazole antifungal agents and allylamine antifungal agents.

In one embodiment, the antifungal agent is present in an amount that is effective at treating antifungal infections. Preferably, the amount is a minimum of about 1 ppm and/or a maximum of about 10 wt. %. More preferably, the amount is a minimum of about 10 ppm, about 100 ppm, about 1000 ppm, about 0.1 wt. % and/or a maximum of about 5 wt. %, about 2 wt. %, about 1 wt. % and about 0.1 wt. %.

The formulation may also comprise additional therapeutic agents including anti-inflammatory agents. Anti-inflammatory agents include but are not limited to steroidal anti-inflammatory agents such as corticosteroids selected from the group consisting of cortisone, dexamethasone, fluorometholone, hydrocortisone, loteprednol, medrysone, methylprednisolone, prednisolone, prednisone, rimexolone, and triamcinolone and non-steroidal anti-inflammatory agents selected from the group consisting of cromolyn, diclofenac, flurbiprofen, ketorolac, Iodoxamide, nedocromil, pemirolast, and suprofen.

Due to the tendency of Alexidine or other biguanide antimicrobial agents to surface bind or hydrolyze in an aqueous solution it is sometimes desirable to include a stabilizer. Alexidine surface binding is a non-specific saturable process. Therefore, the relative loss of alexidine, due to non-specific binding, depends on the absolute concentration of alexidine in solution. For example, 2 ppm alexidine binding to the container surface results in 50% loss for 4 ppm alexidine formulation, but only 2% for 100 ppm formulation. Unlike binding to the surface, acid/base/buffer hydrolysis is a kinetic process. It contributes more significantly to alexidine loss in aqueous solution during storage.

As is understood by one of ordinary skill in the art, a stabilizer is a compound that prevents the diminishing of desirable properties of an active agent in solution. Examples of stabilizers that are effective in an aqueous solution include but are not limited to pharmaceutically acceptable antioxidants (ascorbate, methionine, citric acid, BHT), complexing agents (cyclodextrin and derivatives, hyaluronic acid, citric acid), non-ionic surfactants (poloxamines such as Tetronics® 908, tyloxapol) and chelating agents and salts thereof (hydroxyl alkyl phosphonate, sodium edentate). In one embodiment, preferred stabilizers are hydroxyalkyl phosphonate, ethylenediamine-tetraacetic acid, Tetronics® 908, tyloxapol, cyclodextrin or hyaluronic acid. In one embodiment, the stabilizer is present in an amount effective to stabilize the biguanide antimicrobial agent. An amount effective to stabilize a compound means that the stabilizer is present in an amount that prevents deterioration of at least 90% of the compound in a period of 24 months. In another embodiment, the preferred stabilizer is present in a minimum amount of about 0.001 wt. %, about 0.005 wt. %, about 0.01 wt. % and/or a maximum amount of about 0.5 wt. %, about 0.3 wt. %, about 0.1 wt. %, about 0.08 wt. %, about 0.05 wt. %, about 0.03 wt. % or about 0.01 wt. % based upon the total weight of the composition.

In another embodiment the effective shelf life of the biguanide antimicrobial agent is extended by a minimum of about 10 percent of the shelf life of a comparable composition without the stabilizer. In another embodiment, the shelf life of the biguanide antimicrobial agent is extended by a minimum of about 20 percent, about 40 percent, about 80 percent, about 100 percent or about 200 percent of the shelf life of a comparable composition without a stabilizer.

The invention is provided as a stable solution, suspension, gel, ointment, skin/mucous patch, suppository, etc. such as eye drops, otic solution and nasal solution which contains a biguanide antimicrobial agent which maintains antimicrobial activity when stored.

There are a wide variety of suitable formulations of pharmaceutical compositions of the present invention (See e.g., Remington's Pharmaceutical Sciences, 19th ed., 1995). For example, the compositions of the present invention can be compounded with one or more agents to facilitate their use in a wide variety of contexts. Topical compositions for delivering biguanide antimicrobial agents to the eye according to the present invention will typically comprise the biguanide antimicrobial agent present in a suitable pharmaceutically acceptable carrier. Exemplary pharmaceutically acceptable carriers include, but are not limited to, water, buffered aqueous solutions, isotonic mixtures of water and water-immiscible solvents, such as alkanols, aryl alkanols, vegetable oils, polyalkalene glycols, petroleum-based jellies, ethylcellulose, ethyloleate, carboxymethylcelluloses, polyvinylpyrrolidones, and isopropyl myristates. The compositions of the present invention can also include pharmaceutically acceptable auxiliary components such as buffers, emulsifiers, preservatives, wetting agents, tonicity agents, thixotropic agents, e.g., polyethylene glycols, chelating agents, and additional antimicrobial agents. The compositions of the present invention as formulated are sterile, substantially isotonic, and in full compliance with all Good Manufacturing Practice (GMP) regulations of the U.S. Food and Drug Administration.

In exemplary embodiments of the present invention, pharmaceutically acceptable compositions of the present invention comprising one or more biguanide antimicrobial agents can be aqueous suspensions containing a buffer, (e.g., carbonate salt, phosphate salt, acetate salt, glutamic acid, or citrate salt), an isotonizing agent (e.g., glycerol, mannitol, sorbitol, propylene glycol, sodium chloride, potassium chloride, boric acid), a stabilizer (e.g., sodium edetate, sodium citrate), a non-protein based surfactant (e.g., polysorbate 80, polyoxyethylene(60) hydrogenated castor oil, tyloxapol, benzalkonium chloride, polyoxyethylene fatty acid esters, polyoxyethylene alkylphenyl ethers, and polyoxyethylene alkyl ethers, mixtures thereof), a preservative (e.g., p-hydroxybenzoate and its analogs, benzalkonium chloride, benzethonium chloride, chlorobutanol), a pH control agent (e.g., hydrochloric acid, sodium hydroxide, phosphoric acid), a surfactant polyoxyethylene fatty acid esters, and/or other additives.

The therapeutic agents of the present invention, i.e., biguanide antimicrobial agents, can be incorporated into suitable pharmaceutically acceptable carriers at therapeutically effective concentrations. For treatment purposes, the pharmaceutical formulations of the present invention can be, for example, administered to the subject in a single bolus delivery, via continuous delivery over an extended time period, or in a repeated administration protocol (e.g., by an hourly, daily or weekly, repeated administration protocol). The pharmaceutical formulations of the present invention can be administered, for example, one or more times half-hourly, i.e., every half an hour for a 24 hour period, one or more times hourly, or one or more times daily. In certain embodiments, the pharmaceutical formulations of the invention are administered two times daily, four times daily, six times daily, or twelve times daily. Typically, the formulations are self-administered.

In another embodiment, the composition further comprises a viscosifier.

In yet another embodiment, the viscosifiers are selected from the group consisting of natural polysaccharides, natural gums, synthetic polymers, proteins and synthetic polypeptides that are capable of increasing viscosity and are pharmaceutically acceptable.

In still another embodiment, the viscosifiers are selected from the group consisting of mucomimetics. Preferably, the viscosifier is a carboxyvinyl polymer.

The viscosifiers are optionally used in the present invention to increase the mean residence time of the active ingredient in the eye or other tissue. With the aid of a viscosifier, liquid drops can be used having a viscosity that is a minimum of about 2 cps and a maximum of about 100 cps. Viscosifiers can be used to formulate liquid gels that have a viscosity ranging from about 100 cps to about 1,000 cps. Ophthalmic gels will generally have a viscosity in excess of about 1,000 cps. Regardless, the viscosifier is utilized to ensure an adequate mean residence time in the eye. Any synthetic or natural polymer, which is capable of forming a viscous or a solid insert, may be utilized. In addition to having the physical properties required to form a viscous gel or solid insert, the polymers must also be compatible with tissues of the eye. The polymers must also be chemically and physically compatible with the above-described active agent and other components of the compositions.

Polymers, which satisfy the foregoing criteria, are referred to herein as “pharmaceutically acceptable viscous polymers.” Examples of suitable polymers include: natural polysaccharides and gums, such as alginate, carrageenan, guar, karaya, locust bean, tragacanth and xanthan; synthetic polymers, such as agarose, carboxy vinyl polymer, carboxymethylcellulose, hydroxyethylcel lulose, hydroxypropylcellulose, hydroxypropylmethylcellulose, methylcellulose, polyvinyl alcohol and polyvinyl pyrrolidone.

In addition, proteins and synthetic polypeptides that form viscous gels and are pharmaceutically acceptable can be used to provide better bioavailability. Typically, proteins that can be used include: gelatin, collagen, albumin and casein.

Polymers which have high molecular weights and, most importantly, physical properties which mimic the physical properties of the mucous secretions found in the eye are referred to herein as being “mucomimetic.” Preferred classes of mucomimetic polymers are carboxy vinyl polymers having molecular weights in the range of from about 50,000 to about 6,000,000. The polymers have carboxylic acid functional groups and preferably contain between 2 and 7 carbon atoms per functional group. The gels that form during preparation of the ophthalmic polymer dispersion have a viscosity between about 1,000 to about 300,000 centipoise (cps). Suitable carboxy vinyl polymers include those called carbomers, e.g., Carbopol® (B. F. Goodrich Co., Cleveland, Ohio). Specifically preferred are Carbopol 934, 940, 970, 974 and 980. Such polymers will typically be employed in an amount that is a minimum of about 0.05 wt. % and a maximum of about 8.0 wt. %, depending on the desired viscosity of the composition.

Various anatomical barriers relating to the eye may underlie the poor intraocular penetrance of active ingredients. In this regard, the cornea is the principal barrier to entry of foreign substances. It has two distinct penetration barriers, the corneal epithelium and the corneal stroma. Thus, it is desirable to use a penetration enhancer to improve the penetration of the active ingredients of the present invention.

The penetration enhancer generally acts to make the cell membranes less rigid and therefore more amenable to allowing passage of drug molecules between cells. The penetration enhancers preferably exert their penetration enhancing effect immediately upon application to the eye and maintain this effect for a period of approximately five to ten minutes. The penetration enhancers and any metabolites thereof must also be non-toxic to ophthalmic tissues. One or more penetration enhancers will generally be utilized in a minimum amount of about 0.01 wt. % and/or a maximum of about 10 wt. %. The preferred penetration enhancers are saccharide surfactants, such as dodecylmaltoside (“DDM”), and monoacyl phosphoglycerides, such as lysophosphatidylcholine. The saccharide surfactants and monoacyl phosphoglycerides, which may be utilized, as penetration enhancers in the present invention are known compounds. The use of such compounds to enhance the penetration of ophthalmic drugs is described in U.S. Pat. No. 5,221,696 the entire contents of which are incorporated by reference into the present specification.

Aqueous compositions of the invention have an ophthalmically compatible pH, which generally will range between about 6 to about 8, and more preferably between 6.5 to 7.8, and most preferably about 7 to 7.5. One or more conventional buffers may be employed to obtain the desired pH value. Suitable buffers include for example but are not limited to borate buffers based on boric acid and/or sodium borate, phosphate buffers based on Na₂HPO₄, NaH₂PO₄ and/or KH₂PO₄, citrate buffers based on sodium or potassium citrate and/or citric acid, sodium bicarbonate, aminoalcohol buffers, Good buffers and combinations thereof. Generally, buffers will be used in amounts that are a minimum of about 0.05 wt. % or about 0.1 wt % and a maximum of about 2.5 wt. % or about 1.5 wt. % based upon the total weight of the composition.

Compositions of the present invention likewise include one or more tonicity agents to approximate the osmotic pressure of normal lachrymal fluids, which is equivalent to a 0.9 percent solution of sodium chloride or 2.5 percent glycerin solution. Examples of suitable tonicity agents include but are not limited to sodium and potassium chloride, dextrose, mannose, glycerin, calcium and magnesium chloride. These agents are typically used individually in amounts that are a minimum of about 0.01 wt. % or about 0.2 wt. % and/or a maximum of about 2.5 wt. % or about 1.5 wt. % based upon the total weight of the composition. Preferably, the tonicity agent is employed in an amount to provide a final osmotic value that is a minimum of about 200 mOsm/kg, about 220 mOsm/kg and/or a maximum of about 450 mOsm/kg, about 350 mOsm/kg or about 320 mOsm/kg.

Aqueous compositions may likewise include a humectant to provide moisture to the eye. A first class of humectants is polymer humectants. Examples of suitable humectants include for example but are not limited to poly(vinyl alcohol) (PVA), poly(N-vinylpyrrolidone) (PVP), cellulose derivatives and poly(ethylene glycol). As disclosed in U.S. Pat. No. 6,274,133, cationic cellulosic polymers include for example but are not limited to water soluble polymers commercially available under the CTFA (Cosmetic, Toiletry, and Fragrance Association) designation Polyquaternium-10, including the cationic cellulosic polymers available under the trade name UCARE® Polymers from Amerchol Corp., Edison, N.J., such as for example but not limited to Polymer JR™. Generally, these cationic cellulose polymers contain quaternized N,N-dimethylamino groups along the cellulosic polymer chain.

Another suitable class of humectants is non-polymeric humectants. Examples may include glycerin, propylene glycol, and other non-polymeric diols and glycols. The specific quantities of humectants used in the invention will vary depending upon the application. However, the humectants will typically be included in an amount from about 0.01 or about 0.1 wt. % and/or a maximum of about 5% wt. % or about 2 wt. %.

It will be understood that some constituents possess more than one functional attribute. For example, cellulose derivatives are suitable polymeric humectants, but are also referred to as “viscosity increasing agents” to increase viscosity of the composition if desired. Glycerin is a suitable non-polymeric humectant but is also may contribute to adjusting tonicity.

Compositions of the present invention may optionally include one or more sequestering agents to bind metal ions, which in the case of ophthalmic solutions, might otherwise react with protein deposits and collect on contact lenses. Suitable sequestering agents include for example but are not limited to ethylenediaminetetraacetic acid (EDTA) and its salts. Sequestering agents are preferably present in a minimum of about 0.01 wt. % and/or a maximum of about 0.2 wt. % based upon the total weight of the composition.

Specific Methods for Using the Compositions of the Invention

In one aspect, the compositions of the invention are administered or used topically.

Ophthalmic Care

The physiologically-balanced, solution of the invention may be used in place of a saline solution to remove a foreign body from, to rinse, or to irrigate the eyes. It can also be applied topically before or after surgery to disinfect an eye and surrounding tissues. The solution can be used once or several times a day according to a patient's needs and condition. The solution can be applied by dropping it directly into the eyes as necessary. It can also be applied by soaking gauze and applying the saturated gauze to the eyes for one or several minutes. It can also be used to clean the eyes by gently wiping the eyes with saturated gauze. The solution can also be poured into a small eye washer, then the washer is inverted over the eye and the eyelid opened and closed several times.

The physiologically-balanced, solution of the invention may be used for the treatment of ocular disinfection or decontamination. In addition, it may be used as a replacement for silver nitrate in the disinfection of the eyes of neonates.

The solutions of the present invention may be used for cleaning eyes in adults and in pediatrics. For example, various viral infections, bacterial or fungal infections, or pathogenic agents may be effectively treated with the solution of the present invention.

It will be understood that the present invention is typically applied by administering an aqueous solution to the eye of a patient in the form of eye drops, liquid gels or viscous gels. In one embodiment, one to four drops are applied to each eye. Preferably two drops are applied to each eye. In one embodiment, the drops are placed directly on the eye. In another embodiment, the drops are placed in the conjuntival sac beneath the eye.

Typically, the drops are administered a minimum of once daily, two times daily or three times daily.

Methods of Using a Composition for Skin Disinfection

The composition of the present invention may also be used to treat skin that is infected. In a skin of a patient showing medical signs of infection, the composition of the present invention may be applied directly to the area of the skin that is infected. After at least one application of the composition onto the infected skin using standard methods of application known in the art, the disinfective properties of the composition may be noted.

Methods of Using the Solutions of Invention in Gynecology

The composition of the present invention may be used for the treatment of gynecological infections, such as urinary tract infections and the like. For example, various microorganisms, yeasts (e.g., Monilia, Candida albicans, etc), bacterial infections, HSV-2, HIV or other pathogenic agents may be effectively treated with the solution of the present invention. Optionally, the application of the solutions of the present invention can be used with other medications for the treatment of gynecological infections. For example, use as a lavage of birth canal in pregnant female patients with suspected venereal diseases, and potentially as bathing and cleansing solution on babies right after birth in the delivery rooms of hospitals or as disinfectant on catheters and shunt in dialysis room.

Method of Use as a Treatment for Topical Infections

The compounds of the current invention may be used to treat topical infections by incorporating them into creams, ointments or lotions for use in such conditions. Such creams, ointments or lotions might be used for a broad variety of skin conditions and may incorporate penetration enhancers in order to deliver the antimicrobial activity of the compound to microbes present beneath the outer (epidermis) layers of the skin.

In another embodiment, the solution of the present invention contains a delivery vehicle that increases the mean residence time of the active agent in the eye and/or enhances penetration in the eye U.S. Pat. Nos. 6,884,788 or 6,261,547 or 5,800,807 or 5,618,800 or 5,496,811 disclose various ophthalmic delivery vehicles and are incorporated by reference in their entirety.

According to this invention, there can be obtained a stable composition such as an otic, nasal, vaginal or dermal composition. Other conventional methods can be used unless unsuitable for the object of this invention.

### EXAMPLES

### Example 1

### HSV-1 Viral Suspension Assay

The Viral Suspension Assay was used to evaluate the antiviral properties of Alexidine against Herpes simplex virus type 1 when exposed in suspension for 1, 2, 5, and 10 minutes. The presence of virus (infectivity) was determined by monitoring the virus specific cytopathic effect (CPE) on an appropriate indicator cell line, rabbit kidney. Results are reported as Percent (%) Reduction in virus titer as compared to the corresponding virus control titer (Table 1). The titer of the virus controls were 7.5 log₁₀ following the one minute exposure time; 7.0 log₁₀ following the two minute exposure time; and 7.75 log₁₀ following both the five and ten minute exposure times. The results are listed in Table 1 and show that Alexidine at both 30 ppm and 99 ppm are effective agents against herpes simplex type-1 virus (HSV-1).

TABLE 1
Viral Suspension Assay Percent Reduction of Herpes simplex virus
type 1 after 1, 2, 5 and 10 Minute Exposure to Alexidine

| Alexidine | | | | |
|---|---|---|---|---|
| Test Concentration | 1 minute | 2 minutes | 5 minutes | 10 minutes |
| 30 ppm | 99.99% | 99.99% | 99.9994% | ≧99.99994% |
| 99 ppm | 99.999% | 99.994% | 99.9999% | ≧99.99994% |

### Example 2

### Adenovirus and Cytomegalovirus Testing

The Viral Suspension Assay was used to evaluate the antiviral properties of Alexidine against Adenovirus Type-4, Adenovirus Type-8 and Adenovirus Type-19 and Cytomegalovirus when exposed in suspension for 1, 2, 5, and 10 minutes. The presence of virus (infectivity) was determined by monitoring the virus specific cytopathic effect (CPE) on an appropriate indicator cell line, rabbit kidney. Results are reported as Percent (%) Reduction in virus titer as compared to the corresponding virus control titer (Table 1). The titer of the virus controls were 7.5 log₁₀ following the one minute exposure time; 7.0 log₁₀ following the two minute exposure time; and 7.75 log₁₀ following both the five and ten minute exposure times. The results are listed in Table 2 and show that Alexidine at both 30 ppm and 99 ppm are somewhat effective against viral strains of Adenovirus Type-4, Adenovirus Type-8, and Cytomegalovirus. However, Alexidine did not appear to be effective against the particular strain of Adenovirus Type-19 that was tested. Alexidine is a potent antimicrobial agent against Herpes Simplex-1 and has some effectiveness against certain strains of other viruses that cause ocular infection.

TABLE 2
Viral Suspension Assay Percent Reduction of Adenovirus Type-4,
Adenovirus Type-8 and Adenovirus Type-19 and Cytomegalovirus
after 1, 2, 5 and 10 Minute Exposure to Alexidine

| | Alexidine | Percent Reduction (%) | | | |
|---|---|---|---|---|---|
| | Test | 1 | 2 | 5 | 10 |
| Virus | Concentration | minute | minutes | minutes | minutes |
| Adenovirus | 30 ppm | 43.8 | — | 82.2 | 68.4 |
| type 4 | 99 ppm | 68.4 | — | 43.8 | 68.4 |
| Adenovirus | 30 ppm | 96.8 | 94.4 | 82.2 | 90.0 |
| type 8 | 99 ppm | 82.2 | 82.2 | 90.0 | 90.0 |
| Adenovirus | 30 ppm | No reduction | | | |
| type 19 | 99 ppm | | | | |
| Cytomegalovirus | 30 ppm | 43.8 | 68.4 | — | 43.8 |
| | 99 ppm | 98.2 | 99.0 | 99.8 | 99.98 |

### Example 3—Otic Solution of Alexidine

| Ingredients | % w/w |
|---|---|
| Alexidine | 0.01 |
| HPMC (MV) | 0.5 |
| Mannitol | 3.0 |
| Pluronic F127 | 0.1 |
| EDTA | 0.1 |
| Citrate buffer (0.05M, pH 5.5) | q.s. to 100 |

### Example 4

### Formulation 1

| Ingredient | w/w(%) |
|---|---|
| Alexidine | 30 ppm |
| Nedocromil | 0.02 mg/mL |
| Hydroxylpropyl-beta-cyclodextrin | 5.0 |
| Mannitol | 5.0 |
| PEG 200 | 20.0 |
| Citrate Buffer | pH 5.0 |
| WFI | |

### Example 5

| Ingredient | w/w(%) |
|---|---|
| Anti-glaucoma agent (e.g. brinzolamine) | 0.05 mg/mL |
| Alexidine | 45 ppm |
| HPMC | 0.25 |
| Borate Buffer | pH 6.0 |
| WFI | |

### Example 6

### Formulation 3

| Ingredient | W/W(%) |
|---|---|
| Alexidine | 25 ppm |
| Loteprednol etabonate | 0.02 |
| Mannitol | 5.0 |
| Carbopol | 0.5 |
| EDTA | 0.01 |
| WFI | |

### Example 7

### Formulation 4

| Ingredient | W/W(%) |
|---|---|
| Alexidine | 30 ppm |
| Prednisolone | 0.03 |
| Pluronic F127 | 0.05 |
| CMC | 0.50 |
| Boric Acid | 0.85 |
| Sodium Borate | 0.09 |
| Distill Water | |

What is claimed is:

1. A therapeutic composition comprising a therapeutically effective amount of a biguanide antimicrobial agent in a pharmaceutically acceptable carrier.

2. The composition of claim 1 wherein the biguanide antimicrobial agent is selected from the group consisting of 1,1′-hexamethylene-bis[5-(p-chlorophenyl)biguanide], water soluble salts of 1,1′-hexamethylene-bis[5-(p-chlorophenyl)biguanide], 1,1′-hexamethylene-bis[5-(2-ethylhexyl)biguanide], water-soluble salts of 1,1′-hexamethylene-bis[5-(2-ethylhexyl)biguanide], polyhexamethylene biguanide and combinations thereof.

3. The composition of claim 1 wherein the composition is a topically administrable therapeutic composition for treatment of topical microbial disease.

4. The composition of claim 3 wherein the topical microbial disease is selected from the group consisting of ophthalmic, nasal, otic, vaginal and dermal topical microbial diseases.

5. The composition of claim 1 further comprising an additional antimicrobial agent.

6. The composition of claim 5 wherein the additional antimicrobial agent is present in a synergistically effective amount.

7. The composition of claim 5 wherein the additional antimicrobial agent is selected from the group consisting of antibacterial agents, antiviral agents and antifungal agents.

8. The composition of claim 5 further comprising an additional therapeutic agent.

9. The composition of claim 1 wherein the pharmaceutically acceptable carrier is selected from the group consisting of solids, powders, mixtures of powders, ointments, gels, films, emulsions, suspensions and solutions.

10. The composition of claim 7 wherein the additional antibacterial agent is ISV-403, optical isomers, salt forms, acid forms and/or base forms of ISV-403 as well as mixtures and combinations thereof.

11. The composition of claim 7 wherein the additional antiviral agent is selected from the group consisting of cidofovir, amantadine, rimantadine, acyclovir, ganciclovir, penciclovir, famciclovir, foscarnet, ribavirin, valcyclovir, trifluridine, fomivirsen, vidarabine, cyclosporine, valganciclovir, oseltamivir, adefovir, dipivoxil and zanamivir.

12. The composition of claim 7 wherein the additional antifungal agent is selected from the group consisting of polyene antifungals, imidazole antifungals, triazole antifungals and allylamine antifungals.

13. The composition of claim 1 further comprising a stabilizer.

14. The composition of claim 13 wherein the stabilizer is selected from the group consisting of pharmaceutically acceptable antioxidants, ascorbate, methionine, citric acid, BHT; complexing agents, cyclodextrin and derivatives, hyaluronic acid, citric acid; non-ionic surfactants, poloxamines, tyloxapol; chelating agents and salts thereof, hydroxyl alkyl phosphonate, sodium edentate; and mixtures thereof.

15. The composition of claim 13 wherein the stabilizer is selected from the group consisting of hydroxyalkyl phosphonate, ethylenediamine-tetraacetic acid, Tetronics™ 908, tyloxapol, cyclodextrin, hyaluronic acid and mixtures thereof.

16. The composition of claim 1 wherein the biguanide antimicrobial agent is present between about 1 ppm and about 1 wt. % based upon the total weight of the composition.

17. A method of treating infectious disease comprising topically administering an ophthalmic, otic, nasal, oral, vaginal, dermal composition to a patient, the ophthalmic, otic, nasal, oral, vaginal, dermal composition comprising a biguanide antimicrobial agent and an additional antimicrobial agent in a pharmaceutically acceptable carrier.

18. The method of claim 17, wherein the biguanide antimicrobial agent is selected from the group consisting of 1,1′-hexamethylene-bis[5-(p-chlorophenyl)biguanide], water soluble salts of 1,1′-hexamethylene-bis[5-(p-chlorophenyl)biguanide], 1,1′-hexamethylene-bis[5-(2-ethylhexyl)biguanide], water-soluble salts of 1,1′-hexamethylene-bis[5-(2-ethylhexyl)biguanide], polyhexamethylene biguanide and combinations thereof.

19. The method of claim 17, wherein the composition further comprises a stabilizer.

20. The method of claim 17, wherein the stabilizer is present in an amount effective to extend the shelf life a minimum of about 10% compared to a comparable composition without a stabilizer.

21. The method of claim 17, wherein the biguanide antimicrobial agent is present in an amount that is a minimum of about 1 ppm to a maximum of about 1 wt. % based upon the total weight of the composition.

22. The method of claim 17 wherein the additional antimicrobial agent is selected from the group consisting of antibacterial agents, antiviral agents and antifungal agents.

23. The method of claim 22, wherein the additional antiviral agent is selected from the group comprising of cidofovir, amantadine, rimantadine, acyclovir, ganciclovir, penciclovir, famciclovir, foscarnet, ribavirin, valcyclovir, trifluridine, fomivirsen, vidarabine, cyclosporine, valganciclovir, oseltamivir, adefovir, dipivoxil and zanamivir.

24. The method of claim 23, wherein the additional antiviral agent is present in an amount that is a minimum of about 1 ppm and a maximum of about 10 wt. % based upon the total weight of the composition.

25. A locally administrable ophthalmic, otic, nasal, vaginal or dermal therapeutic composition for the treatment of microbial disease of the eye, ear, nasal passages, vaginal canal or dermis which comprises an effective amount of a stabilized biguanide antimicrobial agent in an amount effective to treat infectious disease and an additional antimicrobial agent in an effective amount.
